(19) United States
(12) Patent Application Publication
(10) Pub. No.: US 2025/0345116 A1
(43) Pub. Date: Nov. 13, 2025

(54) AUTOMATED PREDICTION OF SURGICAL GUIDES USING POINT CLOUDS

(71) Applicant: Howmedica Osteonics Corp., Mahwah, NJ (US)

(72) Inventors: Yannick Morvan, Saint Renan (FR); Jérôme Ogor, Brest (FR); Jean Chaoui, Lyon (FR); Julien Ogor, Brest (FR); Thibaut Nico, Brest (FR)

(21) Appl. No.: 18/872,201

(22) PCT Filed: Jun. 2, 2023
(86) PCT No.: PCT/US2023/024336

§ 371 Date: Dec. 5, 2024

Related U.S. Application Data
(60) Provisional application No. 63/350,785, filed on Jun. 9, 2022.

(51) Int. Cl. A61B 34/10; G06T 7/33; G16H 50/50

(52) U.S. Cl. CPC A61B 34/10; G06T 7/337; G16H 50/50; A61B 2034/104; A61B 2034/105; G06T 2207/10028; G06T 2207/20081; G06T 2207/20084; G06T 2207/30008

(57) ABSTRACT

A method for predicting a tool alignment, the method comprising: obtaining, by a computing system, a first point cloud representing one or more bones of a patient; applying, by the computing system, a point cloud neural network to generate a second point cloud based on the first point cloud, the second point cloud, comprising points indicating the tool alignment; and determining, by the computing system, the tool alignment based on the points indicating the tool alignment.

This application claims priority to U.S. Provisional Patent Application 63/350,785, filed Jun. 9, 2022, the entire content of which is incorporated by reference.

### BACKGROUND

Orthopedic surgeries often involve implanting one or more orthopedic prostheses into a patient. For example, in a total shoulder replacement surgery, a surgeon may attach orthopedic prostheses to a scapula and a humerus of a patient. In an ankle replacement surgery, a surgeon may attach orthopedic prostheses to a tibia and a talus of a patient. When planning an orthopedic surgery, it may be important for the surgeon to select appropriate tool alignment, such as a drilling axis, cutting plane, pin insertion axis, and so on. Selecting an inappropriate tool alignment may lead to improperly limited range of motion, an increased probability of failure of the orthopedic prosthesis, complications during surgery, and other adverse health outcomes.

### SUMMARY

This disclosure describes example techniques for automated prediction of tool alignments and tool alignment guides for orthopedic surgeries. As described in this disclosure, a computing system obtains a first point cloud representing one or more bones of a patient. The computing system may then apply a point cloud neural network to generate a second point cloud based on the first point cloud. In some examples, the second point cloud comprises points indicating the tool alignment. The computing system may determine the tool alignment based on the points indicating the tool alignment. In some examples, the second point cloud comprises points representing a tool alignment guide for aligning a tool during surgery.

In one example, this disclosure describes a method for predicting a tool alignment, the method comprising: obtaining, by a computing system, a first point cloud representing one or more bones of a patient; applying, by the computing system, a point cloud neural network to generate a second point cloud based on the first point cloud, the second point cloud comprising points indicating the tool alignment; and determining, by the computing system, the tool alignment based on the points indicating the tool alignment.

In another example, this disclosure describes a system comprising: a storage system configured to store a first point cloud representing one or more bones of a patient; and processing circuitry configured to: apply a point cloud neural network to generate a second point cloud based on the first point cloud, the second point cloud comprising points indicating a tool alignment; and determine the tool alignment based on the points indicating the tool alignment.

In another example, this disclosure describes a method for predicting a tool alignment guide, the method comprising: obtaining, by a computing system, a first point cloud representing one or more bones of a patient; and applying, by the computing system, a point cloud neural network to generate a second point cloud based on the first point cloud, the second point cloud comprising points representing a tool alignment guide configured to guide a tool along a tool alignment to a target bone of the one or more bones of the patient.

In another example, this disclosure describes a system for predicting a tool alignment guide, the method comprising: storage system configured to store a first point cloud representing one or more bones of a patient; and processing circuitry configured to apply a point cloud neural network to generate a second point cloud based on the first point cloud, the second point cloud comprising points representing a tool alignment guide configured to guide a tool along a tool alignment to a target bone of the one or more bones of the patient.

In other examples, this disclosure describes systems comprising means for performing the methods of this disclosure and computer-readable storage media having instructions stored thereon that, when executed, cause computing systems to perform the methods of this disclosure.

The details of various examples of the disclosure are set forth in the accompanying drawings and the description below. Various features, objects, and advantages will be apparent from the description, drawings, and claims.

### BRIEF DESCRIPTION OF DRAWINGS

FIG. 1 is a block diagram illustrating an example system that may be used to implement the techniques of this disclosure.

FIG. 2 is a block diagram illustrating example components of a planning system, in accordance with one or more techniques of this disclosure.

FIG. 3 is a conceptual diagram illustrating an example point cloud neural network (PCNN), in accordance with one or more techniques of this disclosure.

FIG. 4 is a flowchart illustrating an example architecture of a T-Net model in accordance with one or more techniques of this disclosure.

FIG. 5 is a conceptual diagram illustrating an example 3-dimensional (3D) image representing a predicted tool alignment in accordance with one or more techniques of this disclosure.

FIG. 6 is a conceptual diagram illustrating an example patient-specific guide in accordance with one or more techniques of this disclosure.

FIG. 7 is a flowchart illustrating an example process for predicting a tool alignment in accordance with one or more techniques of this disclosure.

FIG. 8 is a flowchart illustrating an example process for predicting a tool alignment guide in accordance with one or more techniques of this disclosure.

### DETAILED DESCRIPTION

When planning an orthopedic surgery, it may be important for the surgeon to select an appropriate tool alignment, such as a drilling axis, cutting plane, or pin insertion axis. Selecting an inappropriate tool alignment may lead to improper range of motion, an increased probability of failure of the orthopedic prosthesis, complications during surgery, and other adverse health outcomes. Because of the importance of selecting an appropriate tool alignment, planning systems have been developed to help surgeons select orthopedic prostheses. For instance, in some examples, a planning system applies a set of deterministic rules based, e.g., on patient bone geometry, to recommend a tool alignment for a patient. However, the accuracy of such planning system may be deficient, and surgeons may lack confidence in the predictions generated by such automated planning systems. Part of the reason for the deficient accuracy and lack of surgeon confidence is that a surgeon may not be certain that the orthopedic prostheses recommended by the automated planning systems are based on cases similar to the patient that the surgeon is planning to treat. Additional challenges relate to ensuring accuracy of tool alignment guides.

This disclosure describes techniques that may address one or more challenges associated with planning systems for predicting tool alignment. For instance, in accordance with one or more techniques of this disclosure, a computing system may obtain a first point cloud representing one or more bones of a patient. The computing system may apply a point cloud neural network (PCNN) to generate a second point cloud based on the first point cloud. In some examples, the second point cloud comprises points indicating the tool alignment. The computing system may determine the tool alignment based on the second point cloud. The use of point clouds and a PCNN may lead to improved accuracy of tool alignments and tool alignment guides, e.g., because of training the PCNN based on similar patients and experienced surgeons. In some examples, the second point cloud comprises points representing a tool alignment guide for aligning a tool during surgery.

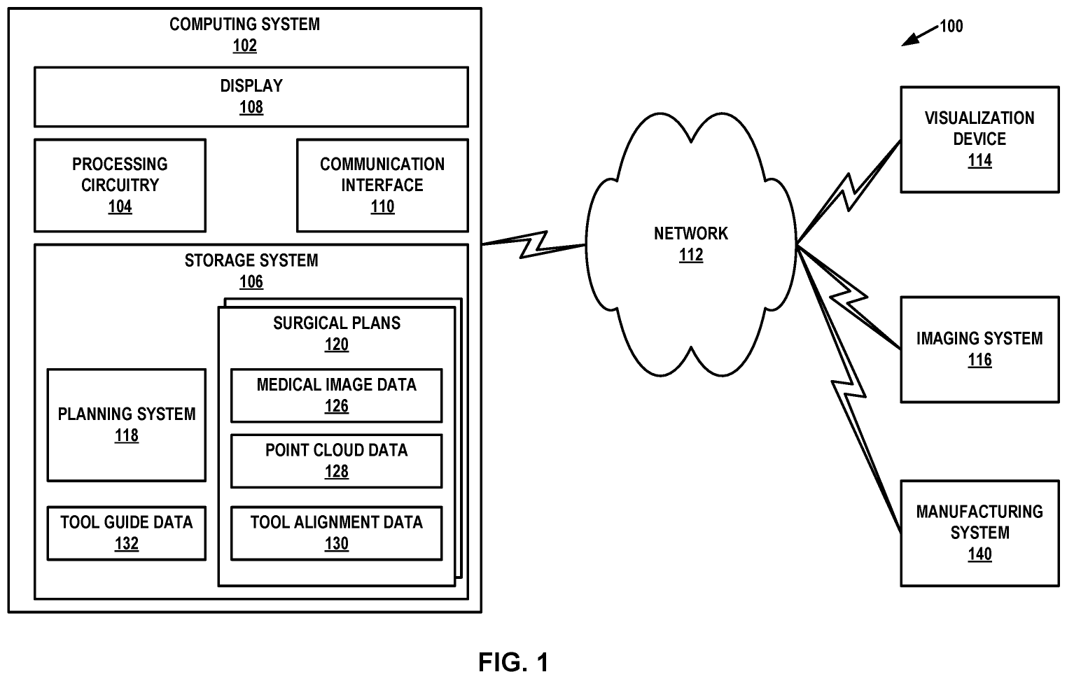

FIG. 1 is a block diagram illustrating an example system 100 that may be used to implement the techniques of this disclosure. FIG. 1 illustrates computing system 102, which is an example of one or more computing devices that are configured to perform one or more example techniques described in this disclosure. Computing system 102 may include various types of computing devices, such as server computers, personal computers, smartphones, laptop computers, and other types of computing devices. In some examples, computing system 102 includes multiple computing devices that communicate with each other. In other examples, computing system 102 includes only a single computing device. Computing system 102 includes processing circuitry 104, storage system 106, a display 108, and a communication interface 110. Display 108 is optional, such as in examples where computing system 102 is a server computer.

Examples of processing circuitry 104 include one or more microprocessors, digital signal processors (DSPs), application specific integrated circuits (ASICs), field programmable gate arrays (FPGAs), discrete logic, software, hardware, firmware or any combinations thereof. In general, processing circuitry 104 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. In some examples, processing circuitry 104 is dispersed among a plurality of computing devices in computing system 102 and visualization device 114. In some examples, processing circuitry 104 is contained within a single computing device of computing system 102.

Processing circuitry 104 may include arithmetic logic units (ALUs), elementary function units (EFUs), digital circuits, analog circuits, and/or programmable cores, formed from programmable circuits. In examples where the operations of processing circuitry 104 are performed using software executed by the programmable circuits, storage system 106 may store the object code of the software that processing circuitry 104 receives and executes, or another memory within processing circuitry 104 (not shown) may store such instructions. Examples of the software include software designed for surgical planning.

Storage system 106 may be formed by any of a variety of memory devices, such as dynamic random access memory (DRAM), including synchronous DRAM (SDRAM), magnetoresistive RAM (MRAM), resistive RAM (RRAM), or other types of memory devices. Examples of display 108 include a liquid crystal display (LCD), a plasma display, an organic light emitting diode (OLED) display, or another type of display device. In some examples, storage system 106 may include multiple separate memory devices, such as multiple disk drives, memory modules, etc., that may be dispersed among multiple computing devices or contained within the same computing device.

Communication interface 110 allows computing system 102 to communicate with other devices via network 112. For example, computing system 102 may output medical images, images of segmentation masks, and other information for display. Communication interface 110 may include hardware circuitry that enables computing system 102 to communicate (e.g., wirelessly or using wires) to other computing systems and devices, such as a visualization device 114 and an imaging system 116. Network 112 may include various types of communication networks including one or more wide-area networks, such as the Internet, local area networks, and so on. In some examples, network 112 may include wired and/or wireless communication links.

Visualization device 114 may utilize various visualization techniques to display image content to a surgeon. In some examples, visualization device 114 is a computer monitor or display screen. In some examples, visualization device 114 may be a mixed reality (MR) visualization device, virtual reality (VR) visualization device, holographic projector, or other device for presenting extended reality (XR) visualizations. For instance, in some examples, visualization device 114 may be a Microsoft HOLOLENS™ headset, available from Microsoft Corporation, of Redmond, Washington, USA, or a similar device, such as, for example, a similar MR visualization device that includes waveguides. The HOLOLENS IM device can be used to present 3D virtual objects via holographic lenses, or waveguides, while permitting a user to view actual objects in a real-world scene, i.e., in a real-world environment, through the holographic lenses. In some examples, there may be multiple visualization devices for multiple users.

Visualization device 114 may utilize visualization tools that are available to utilize patient image data to generate three-dimensional models of bone contours, segmentation masks, or other data to facilitate preoperative planning. These tools may allow surgeons to design and/or select surgical guides and implant components that closely match the patient's anatomy. These tools can improve surgical outcomes by customizing a surgical plan for each patient. An example of such a visualization tool is the BLUEPRINT TM system available from Stryker Corp. The surgeon can use the BLUEPRINT IM system to select, design or modify appropriate implant components, determine how best to position and orient the implant components and how to shape the surface of the bone to receive the components, and design, select or modify tool alignment guide(s) or instruments to carry out the surgical plan. The information generated by the BLUEPRINT TM system may be compiled in a preoperative surgical plan for the patient that is stored in a database at an appropriate location, such as storage system 106, where the preoperative surgical plan can be accessed by the surgeon or other care provider, including before and during the actual surgery.

Imaging system 116 may comprise one or more devices configured to generate medical image data. For example, imaging system 116 may include a device for generating CT images. In some examples, imaging system 116 may include a device for generating MRI images. Furthermore, in some examples, imaging system 116 may include one or more computing devices configured to process data from imaging devices in order to generate medical image data. For example, the medical image data may include a 3D image of one or more bones of a patient. In this example, imaging system 116 may include one or more computing devices configured to generate the 3D image based on CT images or MRI images.

Computing system 102 may obtain a point cloud representing one or more bones of a patient. The point cloud may be generated based on the medical image data generated by imaging system 116. In some examples, imaging system 116 may include one or more computing devices configured to generate the point cloud. Imaging system 116 or computing system 102 may generate the point cloud by identifying the surfaces of the one or more bones in images and sampling points on the identified surfaces. Each point in the point cloud may correspond to a set of 3D coordinates of a point on a surface of a bone of the patient. In other examples, computing system 102 may include one or more computing devices configured to generate the medical image data based on data from devices in imaging system 116.

Storage system 106 of computing system 102 may store instructions that, when executed by processing circuitry 104, cause computing system 102 to perform various activities. For instance, in the example of FIG. 1, storage system 106 may store instructions that, when executed by processing circuitry 104, cause computing system 102 to perform activities associated with a planning system 118. For ease of explanation, rather than discussing computing system 102 performing activities when processing circuitry 104 executes instructions, this disclosure may simply refer to planning system 118 or components thereof as performing the activities or may directly describe computing system 102 as performing the activities.

In the example of FIG. 1, storage system 106 stores surgical plans 120. Surgical plans 120 may correspond to individual patients. A surgical plan corresponding to a patient may include data associated with a planned or completed orthopedic surgery on the corresponding patient. A surgical plan corresponding to a patient may include medical image data 126 for the patient, point cloud data 128, and a tool alignment data 130 for the patient. Medical image data 126 may include computed tomography (CT) images of bones of the patient or 3D images of bones of the patient based on CT images. In this disclosure, the term “bone” may refer to a whole bone or a bone fragment. In some examples, medical image data 126 may include magnetic resonance imaging (MRI) images of one or more bones of the patient or 3D images based on MRI images of the one or more bones of the patient. In some examples, medical image data 126 may include ultrasound images of one or more bones of the patient. Point cloud data 128 may include point clouds representing bones of the patient. Tool alignment data 130 may include data representing one or more tool alignments for use in a surgery. In the example of FIG. 1, storage system 106 may also store tool guide data 132 containing data representing a tool alignment guide. In some examples, tool guide data 132 may be included in surgical plans 120.

Planning system 118 may be configured to assist a surgeon with planning an orthopedic surgery that involves proper alignment of a tool, such as a saw, drill, reamer, punch, or other type of tool. In accordance with one or more techniques of this disclosure, planning system 118 may apply a point cloud neural network (PCNN) to generate an output point cloud based on an input point cloud. Point cloud data 128 may include the input point cloud and/or the output point cloud. The input point cloud represents one or more bones of the patient. In some examples, the output point cloud includes points indicating a tool alignment. Planning system 118 may determine the tool alignment based on the points indicating the tool alignment. In some examples, the output point cloud may include points representing a tool alignment guide configured to guide a tool along a tool alignment to a target bone of the one or more bone of the patient during surgery.

In the example of FIG. 1, system 100 includes a manufacturing system 140. Manufacturing system 140 may manufacture a patient-specific tool alignment guide configured to guide the tool along a tool alignment to the target bone of the one or more bones represented in the input point cloud. For example, manufacturing system 140 may comprise an additive manufacturing device (e.g., a 3D printer) configured to generate the patient-specific tool alignment guide. In other examples, manufacturing system 140 may include other types of devices, such as a reductive manufacturing device, a molding device, or other types of device to generate the patient-specific tool alignment guide.

In an example where the tool alignment corresponds to a cutting plane of an oscillating saw, the patient-specific tool alignment guide may define a slot for an oscillating saw. When the patient-specific tool alignment guide is correctly positioned on a bone of the patient, the slot is aligned with the determined tool alignment. Thus, a surgeon may use the oscillating saw with the determined tool alignment by inserting the oscillating saw into the slot of the patient-specific tool alignment guide. In an example where the tool alignment corresponds to a drilling axis or pin insertion axis, the patient-specific tool alignment guide may define a channel for a drill bit or pin. When the patient-specific tool alignment guide is correctly positioned on a bone of the patient, the channel is aligned with the determined tool alignment. Thus, a surgeon may drill a hole or insert a pin by inserting a drill bit or pin into the channel of the patient-specific tool alignment guide.

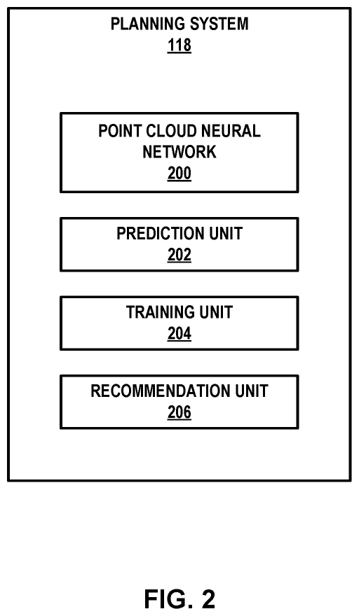

FIG. 2 is a block diagram illustrating example components of planning system 118, in accordance with one or more techniques of this disclosure. In the example of FIG. 2, the components of planning system 118 include a PCNN 200, a prediction unit 202, a training unit 204, and a recommendation unit 206. In other examples, planning system 118 may be implemented using more, fewer, or different components. For instance, training unit 204 may be omitted in instances where PCNN 200 has already been trained. In some examples, one or more of the components of planning system 118 are implemented as software modules. Moreover, the components of FIG. 2 are provided as examples and planning system 118 may be implemented in other ways.

Prediction unit 202 may apply PCNN 200 to generate an output point cloud based on an input point cloud. The input point cloud represents one or more bones of a patient. In some examples, the output point cloud includes points indicating a tool alignment. In some examples, the output point cloud includes points representing a tool alignment guide for aligning a tool during surgery. Prediction unit 202 may obtain the input point cloud in one of a variety of ways. For example, prediction unit 202 may generate the input point cloud based on medical image data (e.g., medical image data 126 of FIG. 1). The medical image data for the patient may include a plurality of input images (e.g., CT images or MRI images, etc.). In this example, each of the input images may have a width dimension and a height dimension, and each of the input images may correspond to a different depth-dimension layer in a plurality of depth-dimension layers. In other words, the plurality of input images may be conceptualized as a stack of 2D images, where the positions of individual 2D images in the stack correspond to the depth dimension. As part of generating the point cloud, prediction unit 202 may perform an edge detection algorithm (e.g., Canny edge detection, Phase Stretch Transform (PST), etc.) on the 2D images (or a 3D image based on the 2D images). Prediction unit 202 may select points on the detected edges as points in the input point cloud. In other examples, prediction unit 202 may obtain the input point cloud from one or devices outside of computing system 102.

As indicated above, the output point cloud may, in some examples, include points indicating a tool alignment. In some such examples, the output point cloud is limited to points indicating the tool alignment. In other words, the output point cloud does not include points representing bone or other tissue of the patient. In some examples, the output point cloud includes points indicating the tool alignment and points representing other objects, such as bones or tissues of the patient. In examples where the tool alignment indicates a cutting plane for an oscillating saw, the points indicating the tool alignment may form a plane oriented and positioned in a coordinate space in a way corresponding to an appropriate alignment of the oscillating saw when cutting a bone. In examples where the tool alignment indicates an insertion axis of a tool (e.g., a drill bit, surgical pin, etc.), the points indicating the tool alignment may form a line oriented and positioned in a coordinate space in a way corresponding to an appropriate alignment of the tool.

In some examples, the output point cloud includes points representing a tool alignment guide for aligning a tool during surgery. In some such examples, the output point cloud is limited to points representing the tool alignment guide. In other words, the output point cloud does not include points representing bone or other tissue of the patient. In some examples, the output point cloud includes points representing the tool alignment guide and points representing other objects, such as bones or tissues of the patient. In some examples where the tool alignment guide includes a slot corresponding to a cutting plane for an oscillating saw, the output point cloud does not include points in locations corresponding to the cutting plane. In examples where the tool alignment guide includes a channel for an insertion axis of a tool (e.g., a drill bit, surgical pin, etc.), the output point cloud does not include points in locations corresponding to the channel.

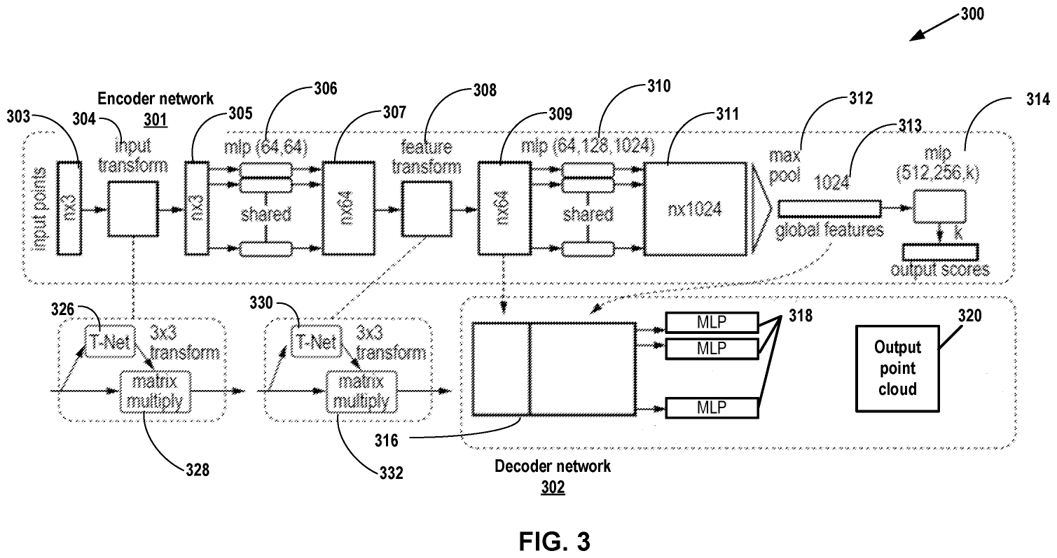

PCNN 200 is implemented using a point cloud learning model-based architecture. A point cloud learning model-based architecture (e.g., a point cloud learning model) is a neural network-based architecture that receives one or more point clouds as input and generates one or more point clouds as output. Example point cloud learning models include PointNet, PointTransformer, and so on. An example point cloud learning model-based architecture based on PointNet is described below with respect to FIG. 3.

Planning system 118 may include different sets of PCNNs for different surgery types. The set of PCNNs for a surgery type may include one or more PCNNs corresponding to different instances where the surgeon aligns a tool with a bone of the patient during a surgery belonging to the surgery type. For example, the set of PCNNs for a total ankle replacement surgery may include a first PCNN that generates an output point cloud that includes points indicating alignments of an oscillating saw when resecting a portion of the patient's distal talus (or points representing a tool alignment guide that defines a slot for aligning an oscillating saw for resection of the portion of the patient's distal talus). In this example, a second PCNN of the set of PCNNs for the total ankle replacement surgery may generate an output point cloud that includes points indicating an axis for inserting a guide pin for attaching a cutting guide (or points representing a tool alignment guide that defines a channel for insertion of the guide pin for attaching a cutting guide).

Training unit 204 may train PCNN 200. For instance, training unit 204 may generate a plurality of training datasets. Each of the training datasets may correspond to a different historic patient in a plurality of historic patients. The historic patients may include patients for whom surgical plans have been developed. For instance, surgical plans 120 (FIG. 1) may include surgical plans for the historic patients. In some examples, the surgical plans may be limited to those developed by expert surgeons, e.g., to ensure high quality training data. In some examples, the historic patients may be selected for relevance. The surgical plans may include data indicating planned tool alignments. For example, a surgical plan may include data indicating that an oscillating saw is to enter a patient's bone at a specific location and at a specific angle. In some examples, the training datasets may include point clouds representing tool alignment guides used during surgeries on historic patients.

The training dataset for a historic patient may include training input data and expected output data. The training input data may include a point cloud representing one or more bones of the patient. In examples where PCNN 200 generates output point clouds indicating tool alignments, the expected output data comprises a point cloud that includes points indicating a tool alignment used during a surgery on the historic patient. In examples where PCNN 200 generates output point clouds representing tool alignment guides, the expected output data may comprise a point cloud that represents a tool alignment guide used during a surgery on the historic patient. In some examples, training unit 204 may generate the training input data based on medical image data stored in surgical plans of historic patients. In some examples, training unit 204 may generate the expected outpoint data based on tool alignments in the surgical plans of historic patients. For instance, the surgical plans of historic patients may include information indicating angles and bone contact positions of tool alignments. Training unit 204 may generate points in the training input point cloud along the indicated angles from the bone contact positions.

In some examples, the surgical plans include post-surgical medical image data. The post-surgical medical image data may be generated after completion of some or all steps of an actual surgery on a historic patient. Training unit 204 may analyze the post-surgical medical image data to determine tool alignments. Training unit 204 may generate training input point clouds based on the determined tool alignments. For example, training unit 204 may determine that an oscillating saw followed a specific cutting plane while resecting a portion of a bone. In this example, training unit 204 may determine a training input point cloud based on the determined cutting plane. In some examples, training unit 204 may receive an indication of user input to indicate areas in the post-surgical medical image data representing portions of the bones that correspond to tool alignments (e.g., planes along which a bone was sawn, holes drilled, etc.). Training unit 204 may sample points within the indicated areas and then fit planes or axes to the sampled points. Training unit 204 may extrapolate these planes or axes away from the bone. Training unit 204 may populate the extrapolated areas of the planes or axes as tool alignments to form a training input point cloud. In some examples where PCNN 200 generates output point clouds representing tool alignment guides, training unit 204 may use the tool alignments determined using PCNN 200 to generate point clouds representing a tool alignment guide. For instance, training unit 204 may generate a tool alignment guide that defines slots or channel corresponding to the determined tool alignments.

Training unit 204 may train PCNN 200 based on the training datasets. Because training unit 204 generates the training datasets based on how real surgeons actually planned and/or executed surgeries in historic patients, a surgeon who ultimately uses a recommendation of a tool alignment or recommendation of a tool alignment guide generated by planning system 118 may have confidence that the recommendation is based on how other real surgeons selected tool alignments or tool alignment guide for real historic patients.

In some examples, as part of training PCNN 200, training unit 204 may perform a forward pass on the PCNN 200 using the input point cloud of a training dataset as input to PCNN 200. Training unit 204 may then perform a process that compares the resulting output point cloud generated by PCNN 200 to the corresponding expected output point cloud. In other words, training unit 204 may use a loss function to calculate a loss value based on the output point cloud generated by PCNN 200 and the corresponding expected output point cloud. In some examples, the loss function is targeted at minimizing a difference between the output point cloud generated by PCNN 200 and the corresponding expected output point cloud. Examples of the loss function may include a Chamfer Distance (CD) and the Earth Mover's Distance (EMD). The CD may be given by the average of a first average and a second average. The first average is an average of distances between each point in the output point cloud generated by PCNN 200 and its closest point in the expected output point cloud. The second average is an average of distances between each point in the expected output point cloud and its closest point in the output point cloud generated by PCNN 200. The CD may be defined as:

$${ℒ}_{C D} ( {S}_{1} , {S}_{2} ) = \frac{1}{2} ( \frac{1}{\vert {S}_{1} \vert} {\sum }_{x ∈ {S}_{1}} {min}_{y ∈ {S}_{2}} ‖ x - y ‖ + \frac{1}{\vert {S}_{2} \vert} {\sum }_{y ∈ {S}_{2}} {min}_{x ∈ {S}_{1}} ‖ x - y ‖ )$$

In the equation above, S₁ is the output point cloud generated by PCNN 200, S₂ is the expected output point cloud, | . . . | is an element indicating number of elements, and ∥ . . . ∥ indicates absolute value.

Training unit 204 may then perform a backpropagation process based on the loss value to adjust parameters of PCNN 200 (e.g., weights of neurons of PCNN 200). In some examples, training unit 204 may determine an average loss value based on loss values calculated from output point clouds generated by performing multiple forward passes through PCNN 200 using different input point clouds of the training data. In such examples, training unit 204 may perform the backpropagation process using the average loss value to adjust the parameters of PCNN 200. Training unit 204 may repeat this process during multiple training epochs.

During use of PCNN 200 (e.g., after training of PCNN 200), prediction unit 202 of planning system 118 may apply PCNN 200 to generate an output point cloud for a patient based on an input point cloud representing one or more bones of the patient. In some examples, recommendation unit 206 may determine a tool alignment based on the output point cloud. For instance, in examples where the tool alignment corresponds to a cutting plane, the points of the output point cloud might not be perfectly positioned within the cutting plane. In such examples, recommendation unit 206 may determine the tool alignment by fitting a plane to the points in the output point cloud indicating the tool alignment. In examples where the tool alignment corresponds to a tool insertion axis (e.g., a drilling axis, pin insertion axis, etc.), the points of the output point cloud might not be perfectly aligned in the tool insertion axis. Accordingly, in such examples, recommendation unit 206 may fit a line (e.g., using a regression process) to the points of the output point cloud representing the tool alignment.

In some examples, recommendation unit 206 may determine a tool alignment guide based on the output point cloud. For example, recommendation unit 206 may perform a 3D reconstruction algorithm, such as a Poisson reconstruction algorithm or a Point2Mesh CNN, to generate a 3D mesh based on the output point cloud. The 3D reconstruction algorithm may generate the 3D mesh at least in part by deforming a template input guide mesh to fit the points of the output point cloud. In some examples, prior to performing the 3D reconstruction algorithm, recommendation unit 206 may register the output point cloud with a model of one or more bones of the patient (e.g., a model based on the input point cloud or a model on which the input point cloud is based) with the output point cloud. Recommendation unit 206 may then exclude from the output point cloud any points of the output point cloud that are internal to the bone model.

In some examples, such as examples where the output point cloud represents the tool alignment guide, recommendation unit 206 may determine one or more parameters of the tool alignment guide based on the output point cloud. The parameters of the tool alignment guide may characterize the tool alignment guide so that the tool alignment guide may be selected or manufactured based on the parameters of the tool alignment guide. For example, recommendation unit 206 may determine a width of the tool alignment guide, curvature of arms of the tool alignment guide, and so on. Recommendation unit 206 may determine the width of the tool alignment guide based on a distance between lateral-most and medial-most points in the output point cloud. Recommendation unit 206 may determine the curvature of the arms of the tool alignment guide by applying a regression to points corresponding to the arms.

In some examples, recommendation unit 206 may output for display one or more images (e.g., one or more 2D or 3D images) or models showing the tool alignment. For example, recommendation unit 206 may output for display an image showing the tool alignment relative to models of the one or more bones of the patient. In some such examples, the output point cloud generated by PCNN 200 and the input point cloud (which represents one or more bones of the patient) are in the same coordinate system. Accordingly, recommendation unit 206 may position the tool alignment determined by recommendation unit 206 based on the output point cloud within the coordinate system of the input point cloud. Recommendation unit 206 may then reconstruct a bone model from the points of the input point cloud (e.g., by using points of the input point cloud as vertices of polygons, where the polygons form a hull of the bone model). In some examples, recommendation unit 206 may output for display one or more images or models showing a tool alignment guide.

In some examples, recommendation unit 206 may generate, based on the output point cloud, a MR visualization indicating the tool alignment. In examples where visualization device 114 (FIG. 1) is an MR visualization device, visualization device 114 may display the MR visualization. In some examples, visualization device 114 may display the MR visualization during a planning phase of a surgery. In such examples, recommendation unit 206 may generate the MR visualization as a 3D image in space. Recommendation unit 206 may generate the 3D image in the same as described above for generating the 3D image. In some examples, recommendation unit 206 may generate, based on the output point cloud, an MR visualization of a tool alignment guide.

In some examples, the MR visualization is an intra-operative MR visualization. In other words, visualization device 114 may display the MR visualization during surgery. In some examples, visualization device 114 may perform a registration process that registers the MR visualization with the physical bones of the patient. Accordingly, in such examples, a surgeon wearing visualization device 114 may be able to see the tool alignment relative to a bone of the patient. For example, the surgeon may see a virtual cutting plane extending away from the patient's bone along the determined tool alignment. In another example, the surgeon may see a virtual drilling axis extending away from the patient's bone along the determined tool alignment. This may enable the surgeon to use a tool (e.g., oscillating saw, drill, etc.) without the use of a physical patient-specific tool alignment guide. In some examples where the output point cloud represents a tool alignment guide, recommendation unit 206 may generate, based on the output point cloud, a MR visualization representing the tool alignment guide during surgery.

In some examples, computing system 102 may control operation of a tool based on alignment of the tool with a determined tool alignment. For example, visualization device 114 may perform registration processes to relate the locations of the tool, bone, and tool alignment with one another. During a phase of the surgery in which a surgeon is to use the tool at the determined tool alignment, computing system 102 may determine whether the tool is aligned with the determined tool alignment. If the tool is not aligned with the determined tool alignment, computing system 102 may communicate with the tool to prevent the tool from operating. For example, computing system 102 may prevent the tool from operating if a deviation of the tool from the tool alignment is greater than 1-degree or displaced by more than 1 millimeter.

FIG. 3 is a conceptual diagram illustrating an example point cloud learning model 300 in accordance with one or more techniques of this disclosure. Point cloud learning model 300 may receive an input point cloud. The input point cloud is a collection of points. The points in the collection of points are not necessarily arranged in any specific order. Thus, the input point cloud may have an unstructured representation.

In the example of FIG. 3, point cloud learning model 300 includes an encoder network 301 and a decoder network 302. Encoder network 301 receives an array 303 of n points. The points in array 303 may be the input point cloud of point cloud learning model 300. In the example of FIG. 3, each of the points in array 303 has a dimensionality of 3. For instance, in a Cartesian coordinate system, each of the points may have an x coordinate, a y coordinate, and a z coordinate.

Encoder network 301 may apply an input transform 304 to the points in array 303 to generate an array 305. Encoder network 301 may then use a first shared multi-layer perceptron (MLP) 306 to map each of the n points in array 305 from three dimensions to a larger number of dimensions a (e.g., a=64 in the example of FIG. 3), thereby generating an array 307 of n x a (e.g., n x 64 values). For ease of explanation, the following description of FIG. 3 assumes that a is equal to 64 but in other examples other values of a may be used. Encoder network 301 may then apply a feature transform 308 to the values in array 307 to generate an array 309 of n x 64 values. For each of the n points in array 309, encoder network 301 uses a second shared MLP 310 to map the n points from a dimension to b dimensions (e.g., b=1024 in the example of FIG. 3), thereby generating an array 311 of n x b (e.g., n x 1024 values). For ease of explanation, the following description of FIG. 3 assumes that b is equal to 1024 but in other examples other values of b may be used. Encoder network 301 applies a max pooling layer 312 to generate a global feature vector 313. In the example of FIG. 3, each of points n in global feature vector 313 has 1024 dimensions.

Thus, as part of applying an PCNN 200, computing system 102 may apply an input transform (e.g., input transform 304) to a first array (e.g., array 303) that comprises the point cloud to generate a second array (e.g., array 305), wherein the input transform is implemented using a first T-Net model (e.g., T-Net Model 326), apply a first MLP (e.g., MLP 306) to the second array to generate a third array (e.g., array 307), apply a feature transform (e.g., feature transform 308) to the third array to generate a fourth array (e.g., array 309), wherein the input transform is implemented using a second T-Net model (e.g., T-Net model 330), apply a second MLP (e.g., MLP 310) to the fourth array to generate a fifth array (e.g., array 311); and apply a max pooling layer (e.g., max pooling layer 312) to the fifth array to generate the global feature vector (e.g., global feature vector 313).

A fully-connected network 314 may map global feature vector 313 to k output classification scores. The value k is an integer indicating a number of classes. Each of the output classification scores corresponds to a different class. An output classification score corresponding to a class may indicate a level of confidence that the input point cloud as a whole corresponds to the class. Fully-connected network 314 includes a neural network having two or more layers of neurons in which each neuron in a layer is connected to each neuron in a subsequent layer. In the example of FIG. 3, fully-connected network 314 includes an input layer having 512 neurons, a middle layer having 256 neurons, and an output layer having k neurons. In some examples, fully-connected network 314 may be omitted from encoder network 301.

In some examples, input 316 to decoder network 302 may be formed by concatenating the n 64-dimensional points of array 309 with global feature vector 313. In other words, for each point of the n points in array 309, the corresponding 64 dimensions of the point are concatenated with the 1024 features in global feature vector 313. In some examples, array 309 is not concatenated with global feature vector 313.

Decoder network 302 may sample N points in a unit square in 2-dimensions. Thus, decoder network 302 may randomly determine N points having x-coordinates in a range of [0,1] and y-coordinates in the range of [0,1]. For each respective point of the N points, decoder network 302 may obtain a respective input vector by concatenating the respective point with global feature vector 313. Thus, in examples where array 309 is not concatenated with global feature vector 313, each of the input vectors may have 1026 features. For each respective input vector, decoder network 302 may apply each of K MLPs 318 (where K is an integer greater than or equal to 1) to the respective input vector. Each of MLPs 318 may correspond to a different patch (e.g., area) of the output point cloud. When decoder network 302 applies the MLP to an input vector, the MLP may generate a 3-dimensional point in the patch (e.g., area) corresponding to the MLP. Thus, each of the MLPs 318 may reduce the number of features from 1026 to 3. The 3 features may correspond to the 3 coordinates of a point of the output point cloud. For instance, for each sampled point n in N, the MLPs 318 may reduce the features from 1026 to 512 to 256 to 128 to 64 to 3. Thus, decoder network 302 may generate a K×N×3 vector containing an output point cloud 320. In some examples, K=16 and N=512, resulting in second point cloud with 8192 3D points. In other examples, other values of K and N may be used. In some examples, as part of training the MLPs of decoder network 302, decoder network 302 may calculate a chamfer loss of an output point cloud relative to a ground-truth point cloud. Decoder network 302 may use the chamfer loss in a backpropagation process to adjust parameters of the MLPs. In this way, planning system 118 may apply the decoder (e.g., decoder network 302) to generate the premorbid bone model based on the global feature vector.

In some examples, MLPs 318 may include a series of four fully-connected layers of neurons. For each of MLPs 318, decoder network 302 may pass an input vector of 1026 features to an input layer of the MLP. The fully-connected layers may reduce to number of features from 1026 to 512 to 256 to 3.

Input transform 304 and feature transform 308 in encoder network 301 may provide transformation invariance. In other words, point cloud learning model 300 may be able to generate output point clouds (e.g., output bone models) in the same way, regardless of how the input point cloud (e.g., input bone model) is rotated, scaled, or translated. The fact that point cloud learning model 300 provides transform invariance may be advantageous because it may reduce the susceptibility of point cloud learning model 300 to errors based on positioning/scaling in morbid bone models. As shown in the example of FIG. 3, input transform 304 may be implemented using a T-Net Model 326 and a matrix multiplication operation 328. T-Net Model 326 generates a 3×3 transform matrix based on array 303. Matrix multiplication operation 328 multiplies array 303 by the 3×3 transform matrix. Similarly, feature transform 308 may be implemented using a T-Net model 330 and a matrix multiplication operation 332. T-Net model 330 may generate a 64×64 transform matrix based on array 307. Matrix multiplication operation 328 multiplies array 307 by the 64×64 transform matrix.

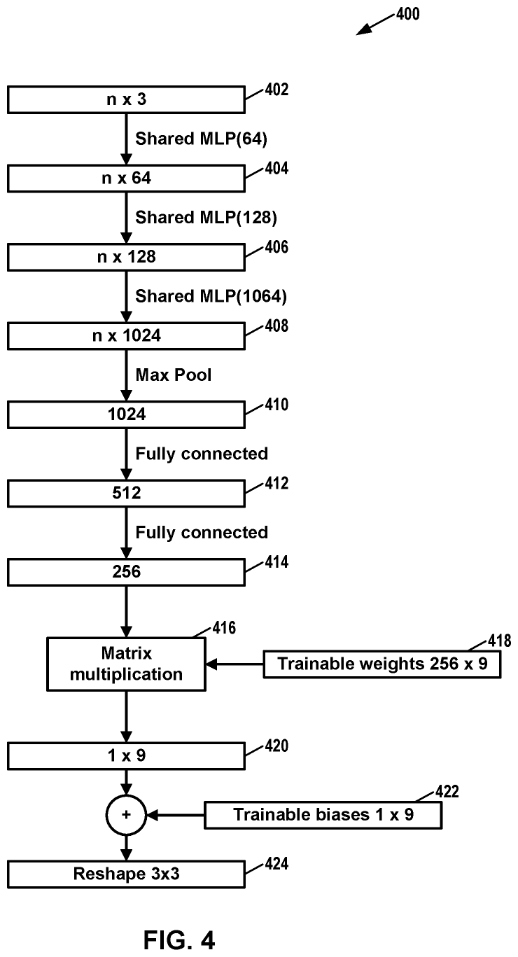

FIG. 4 is a block diagram illustrating an example architecture of a T-Net model 400 in accordance with one or more techniques of this disclosure. T-Net model 400 may implement T-Net Model 326 used in the input transform 304. In the example of FIG. 4, T-Net model 400 receives an array 402 as input. Array 402 includes n points. Each of the points has a dimensionality of 3. A first shared MLP maps each of the n points in array 402 from 3 dimensions to 64 dimensions, thereby generating an array 404. A second shared MLP maps each of the n points in array 404 from 64 dimensions to 128 dimensions, thereby generating an array 406. A third shared MLP maps each of the n points in array 406 from 128 dimensions to 1024 dimensions, thereby generating an array 408. T-Net model 400 then applies a max pooling operation to array 408, resulting in an array 810 of 1024 values. A first fully-connected neural network maps array 410 to an array 812 of 512 values. A second fully-connected neural network maps array 412 to an array 414 of 256 values. T-Net model 400 applies a matrix multiplication operation 416 to a matrix of trainable weights 418. The matrix of trainable weights 418 has dimensions of 256×9. Thus, multiplying array 414 by the matrix of trainable weights 418 results in an array 820 of size 1×9. T-Net model 400 may then add trainable biases 422 to the values in array 420. A reshaping operation 424 may remap the values resulting from adding trainable biases 422 into a 3×3 transform matrix. In other examples, the sizes of the matrixes and arrays may be different.

T-Net model 330 (FIG. 3) may be implemented in a similar way as T-Net model 400 in order to perform feature transform 308. However, in this example, the matrix of trainable weights 418 is 256×4096 and the trainable biases 422 has size 1×4096 bias values instead of 9. Thus, the T-Net model for performing feature transform 308 may generate a transform matrix of size 64×64. In other examples, the sizes of the matrixes and arrays may be different.

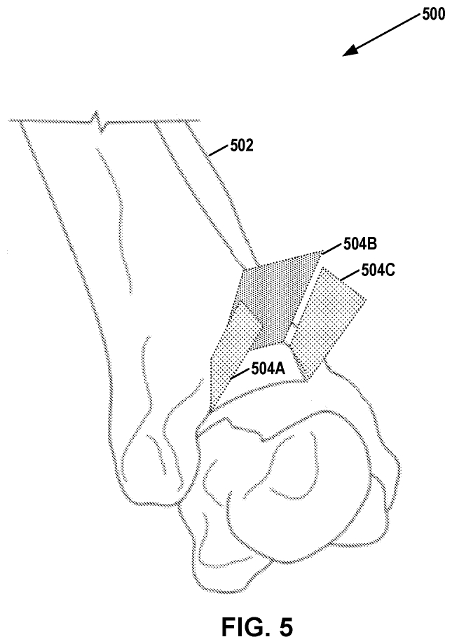

FIG. 5 is a conceptual diagram illustrating an example 3D image 500 representing a predicted tool alignment in accordance with one or more techniques of this disclosure. 3D image 500 shows a distal tibia 502 of a patient. In addition, 3D image 500 shows three tool alignments 504A, 504B, and 504C (collectively, “tool alignments 504”). Tool alignments 504 represent cutting planes for resecting a section of the distal tibia 502 as part of a total ankle replacement surgery. Planning system 118 may obtain a point cloud representing distal tibia 502. Additionally, prediction unit 202 of planning system 118 may apply PCNN 200 to generate one or more output point clouds indicating tool alignments 504. Recommendation unit 206 of planning system 118 may determine tool alignments 504 based on the output point clouds generated by PCNN 200.

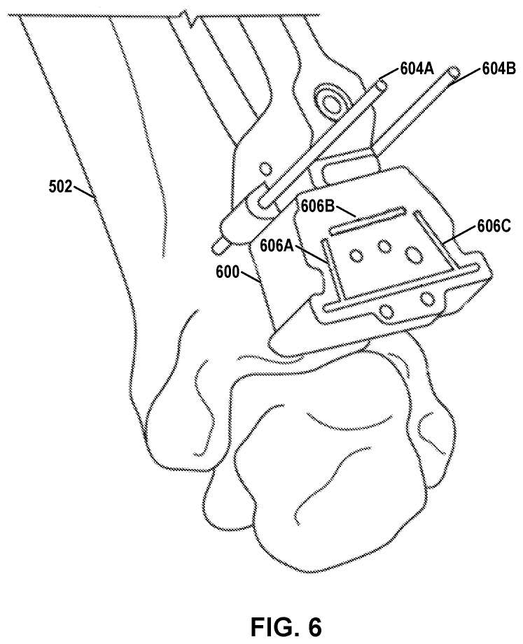

FIG. 6 is a conceptual diagram illustrating an example patient-specific guide 600 in accordance with one or more techniques of this disclosure. In the example of FIG. 6, patient-specific guide 600 is attached to distal tibia 502 of the patient using guide pins 604A, 604B (collectively, “guide pins 604”). Patient-specific guide 600 defines slots 606A, 606B, and 606C (collectively, “slots 606”). Slots 606 are aligned with tool alignments 504 of FIG. 5. Thus, during a surgery, a surgeon may use an oscillating saw to cut distal tibia 502 along tool alignments 504 by inserting an oscillating saw into slots 606. In some examples, patient-specific guide 600 may be manufactured based on predicted tool alignment. In some examples, PCNN 200 may generate a point cloud representing patient-specific guide 600.

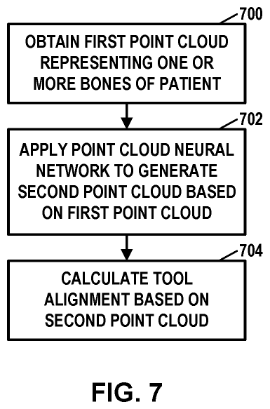

FIG. 7 is a flowchart illustrating an example process for predicting a tool alignment in accordance with one or more techniques of this disclosure. In the example of FIG. 7, computing system 102 may obtain a first point cloud representing one or more bones of a patient (700). In some examples, computing system 102 may obtain the first point cloud by generating the first point cloud based on one or more medical images. In some examples, computing system 102 may obtain the first cloud by receiving the first point cloud from one or more other computing devices or systems.

Additionally, computing system 102 may apply PCNN 200 to generate a second point cloud based on the first point cloud, the second point cloud comprising points indicating the tool alignment (702). For example, computing system 102 may perform a forward pass through PCNN 200 using the first input point cloud as input to an input layer of PCNN 200. An output layer of PCNN 200 outputs the second point cloud. In some examples, the second point cloud may include points representing a target bone of the patient (i.e., a bone to be affected by use of the tool) and the points indicating the tool alignment.

Computing system 102 may determine the tool alignment based on the points indicating the tool alignment (704). For example, computing system 102 may fit a plane or line to the points indicating the tool alignment. The tool alignment corresponds to the fitted plane or line. In some examples, to ease fitting of the plane or line, computing system 102 may remove outlier points from the second point cloud. Outlier points may be points having distances from closest neighboring points of greater than a particular amount. The particular amount may be defined in terms of a multiplier of a standard deviation of the distances between points and their closest neighbors.

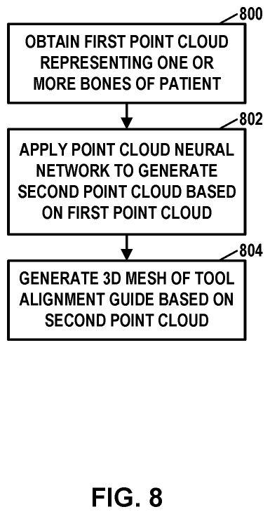

FIG. 8 is a flowchart illustrating an example process for predicting a tool alignment guide in accordance with one or more techniques of this disclosure. In the example of FIG. 8, computing system 102 may obtain a first point cloud representing one or more bones of a patient (800). In some examples, computing system 102 may obtain the first point cloud by generating the first point cloud based on one or more medical images. In some examples, computing system 102 may obtain the first cloud by receiving the first point cloud from one or more other computing devices or systems.

Additionally, computing system 102 may apply PCNN 200 to generate a second point cloud based on the first point cloud, the second point cloud comprising points representing a tool alignment guide configured to guide a tool (e.g., drill bit, pin, oscillating saw, etc.) along a tool alignment to a target bone of the one or more bones of the patient (802). For example, computing system 102 may perform a forward pass through PCNN 200 using the first input point cloud as input to an input layer of PCNN 200. An output layer of PCNN 200 outputs the second point cloud. In some examples, the second point cloud may include points representing a target bone of the patient (i.e., a bone to be affected by use of the tool) and the points representing the tool alignment guide. In some such examples, the spatial arrangement of the points representing the target bone and the points representing the tool alignment guide may indicate an appropriate positioning of the tool alignment guide and the target bone during use of the tool alignment guide. The tool alignment guide may be configured to guide the tool along one or more of a cutting plane, a drilling axis, or a pin insertion axis.

In the example of FIG. 8, computing system 102 may generate a 3D mesh of the tool alignment guide based on the second point cloud (804). For example, computing system 102 may generate the 3D mesh at least in part by deforming a template input guide mesh to fit the points of the second point cloud. After generating the 3D mesh of the tool alignment guide, the 3D mesh may be used as basis for manufacturing the tool alignment guide, e.g., using an additive manufacturing process such as 3D printing. In other examples, computing system 102 does not generate the 3D mesh of the tool alignment guide, but may use the second point cloud for other purposes.

The following is a non-limiting list of clauses in accordance with one or more techniques of this disclosure.

Clause 1. A method for predicting a tool alignment, the method comprising: obtaining, by a computing system, a first point cloud representing one or more bones of a patient; applying, by the computing system, a point cloud neural network to generate a second point cloud based on the first point cloud, the second point cloud comprising points indicating the tool alignment; and determining, by the computing system, the tool alignment based on the points indicating the tool alignment.

Clause 2. The method of clause 1, wherein the tool alignment is one of: a cutting plane, a drilling axis, or a pin insertion axis.

Clause 3. The method of any of clauses 1-2, further comprising manufacturing a patient-specific tool alignment guide configured to guide a tool along the tool alignment to a target bone of the one or more bones of the patient.

Clause 4. The method of any of clauses 1-3, further comprising generating, by the computing system, based on the second point cloud, a Mixed Reality visualization indicating the tool alignment.

Clause 5. The method of any of clauses 1-4, wherein the method further comprises controlling, by the computing system, operation of a tool based on alignment of the tool with the tool alignment.

Clause 6. The method of any of clauses 1-5, wherein the second point cloud includes points representing a target bone from the one or more bones of the patient and the points indicating the tool alignment.

Clause 7. The method of any of clauses 1-6, wherein determining the tool alignment based on the second point cloud comprises fitting a line or plane to a set of points in the second point cloud.

Clause 8. The method of any of clauses 1-7, wherein applying the point cloud neural network comprises: applying an input transform to a first array that comprises the first point cloud to generate a second array, wherein the input transform is implemented using a first T-Net model; applying a first multi-layer perceptron (MLP) to the second array to generate a third array; applying a feature transform to the third array to generate a fourth array, wherein the input transform is implemented using a second T-Net model; applying a second MLP to the fourth array to generate a fifth array; applying a max pooling layer to the fifth array to generate a global feature vector; sampling N points in a unit square in 2-dimensions; concatenating the sampled points with the global feature vector to obtain a combined vector; and applying one or more third MLPs to generate points in the second point cloud.

Clause 9. The method of any of clauses 1-8, further comprising training the PCNN, wherein training the PCNN comprises: generating training datasets based on surgical plans of historic patients; and training the PCNN using the training datasets.

Clause 10. A system comprising: a storage system configured to store a first point cloud representing one or more bones of a patient; and processing circuitry configured to: apply a point cloud neural network to generate a second point cloud based on the first point cloud, the second point cloud comprising points indicating a tool alignment; and determine the tool alignment based on the points indicating the tool alignment.

Clause 11. The system of clause 10, wherein the tool alignment is one of: a cutting plane, a drilling axis, or a pin insertion axis.

Clause 12. The system of any of clauses 10-11, further comprising a manufacturing system configured to manufacture a patient-specific tool alignment guide configured to guide a tool along the tool alignment to a target bone of one or more bones of the patient.

Clause 13. The system of any of clauses 10-12, wherein the processing circuitry is further configured to generate, based on the second point cloud, a Mixed Reality visualization indicating the tool alignment.

Clause 14. The system of any of clauses 10-13, wherein the processing circuitry is further configured to control operation of a tool based on alignment of the tool with the tool alignment.

Clause 15. The system of any of clauses 10-14, wherein the second point cloud includes points representing a target bone of the one or more bones of the patient and the points indicating the tool alignment.

Clause 16. The system of any of clauses 10-15, wherein the processing circuitry is configured to, as part of determining the tool alignment based on the second point cloud, fit a line or plane to a set of points in the second point cloud.

Clause 17. The system of any of clauses 10-16, wherein the processing circuitry is configured to, as part of applying the point cloud neural network: apply an input transform to a first array that comprises the first point cloud to generate a second array, wherein the input transform is implemented using a first T-Net model; apply a first multi-layer perceptron (MLP) to the second array to generate a third array; apply a feature transform to the third array to generate a fourth array, wherein the input transform is implemented using a second T-Net model; apply a second MLP to the fourth array to generate a fifth array; apply a max pooling layer to the fifth array to generate a global feature vector; sample N points in a unit square in 2-dimensions; concatenate the sampled points with the global feature vector to obtain a combined vector; and apply one or more third MLPs to generate points in the second point cloud.

Clause 18. The system of any of clauses 10-17, wherein the processing circuitry is further configured to train the point cloud neural network, wherein the processing circuitry is configured to, as part of training the PCNN: generate training datasets based on surgical plans of historic patients; and train the PCNN using the training datasets.

Clause 19. A method for predicting a tool alignment guide, the method comprising: obtaining, by a computing system, a first point cloud representing one or more bones of a patient; and applying, by the computing system, a point cloud neural network to generate a second point cloud based on the first point cloud, the second point cloud comprising points representing a tool alignment guide configured to guide a tool along a tool alignment to a target bone of the one or more bones of the patient.

Clause 20. The method of clause 19, wherein the tool alignment guide is configured to guide the tool along one of: a cutting plane, a drilling axis, or a pin insertion axis.

Clause 21. The method of any of clauses 19-20, further comprising manufacturing the tool alignment guide.

Clause 22. The method of any of clauses 19-21, further comprising generating, by the computing system, based on the second point cloud, a Mixed Reality visualization indicating the tool alignment guide.

Clause 23. The method of any of clauses 19-22, wherein the second point cloud includes points representing the target bone and the points representing the tool alignment guide.

Clause 24. The method of any of clauses 19-23, wherein applying the point cloud neural network to generate the second point cloud comprises: applying an input transform to a first array that comprises the first point cloud to generate a second array, wherein the input transform is implemented using a first T-Net model; applying a first multi-layer perceptron (MLP) to the second array to generate a third array; applying a feature transform to the third array to generate a fourth array, wherein the input transform is implemented using a second T-Net model; applying a second MLP to the fourth array to generate a fifth array; applying a max pooling layer to the fifth array to generate a global feature vector; sampling N points in a unit square in 2-dimensions; concatenating the sampled points with the global feature vector to obtain a combined vector; and applying one or more third MLPs to generate points in the second point cloud.

Clause 25. The method of any of clauses 19-24, further comprising training the PCNN, wherein training the PCNN comprises: generating training datasets based on surgical plans of historic patients; and training the PCNN using the training datasets.

Clause 26. A system for predicting a tool alignment guide, the system comprising: a storage system configured to store a first point cloud representing one or more bones of a patient; and processing circuitry configured to apply a point cloud neural network to generate a second point cloud based on the first point cloud, the second point cloud comprising points representing a tool alignment guide configured to guide a tool along a tool alignment to a target bone of the one or more bones of the patient.

Clause 27. The system of clause 26, wherein the tool alignment guide is configured to guide the tool along one of: a cutting plane, a drilling axis, or a pin insertion axis.

Clause 28. The system of any of clauses 26-27, further comprising a manufacturing system configured to manufacture the tool alignment guide.

Clause 29. The system of any of clauses 26-28, wherein the processing circuitry is further configured to generate, based on the second point cloud, a Mixed Reality visualization indicating the tool alignment guide.

Clause 30. The system of any of clauses 26-29, wherein the second point cloud includes points representing the target bone from the one or more bones of the patient and the points representing the tool alignment guide.

Clause 31. The system of any of clauses 26-30, wherein the processing circuitry is configured to, as part of applying the point cloud neural network to generate the second point cloud: apply an input transform to a first array that comprises the first point cloud to generate a second array, wherein the input transform is implemented using a first T-Net model; apply a first multi-layer perceptron (MLP) to the second array to generate a third array; apply a feature transform to the third array to generate a fourth array, wherein the input transform is implemented using a second T-Net model; apply a second MLP to the fourth array to generate a fifth array; apply a max pooling layer to the fifth array to generate a global feature vector; sample N points in a unit square in 2-dimensions; concatenate the sampled points with the global feature vector to obtain a combined vector; and apply one or more third MLPs to generate points in the second point cloud.

Clause 32. The system of any of clauses 26-31, wherein the processing circuitry is further configured to train the PCNN, wherein the processing circuitry is configured to, as part of training the PCNN: generate training datasets based on surgical plans of historic patients; and train the PCNN using the training datasets.

Clause 33. A system comprising means for performing the methods of any of clauses 1-9 or 19-25.

Clause 34. One or more non-transitory computer-readable storage media having instructions stored thereon that, when executed, cause a computing system to perform the methods of any of clauses 1-9 or clauses 19-25.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuitry,” as used herein may refer to any of the foregoing structures or any other structure suitable for implementation of the techniques described herein.

1. A method for predicting a tool alignment, the method comprising:
obtaining, by a computing system, a first point cloud representing one or more bones of a patient;
applying, by the computing system, a point cloud neural network to generate a second point cloud based on the first point cloud, the second point cloud comprising points indicating the tool alignment; and
determining, by the computing system, the tool alignment based on the points indicating the tool alignment.

2. The method of claim 1, wherein the tool alignment is one of: a cutting plane, a drilling axis, or a pin insertion axis.

3. The method of claim 1, further comprising manufacturing a patient-specific tool alignment guide configured to guide a tool along the tool alignment to a target bone of the one or more bones of the patient.

4. The method of claim 1, further comprising generating, by the computing system, based on the second point cloud, a Mixed Reality visualization indicating the tool alignment.

5. The method of claim 1, wherein the method further comprises controlling, by the computing system, operation of a tool based on alignment of the tool with the tool alignment.

6. The method of claim 1, wherein the second point cloud includes points representing a target bone from the one or more bones of the patient and the points indicating the tool alignment.

7. The method of claim 1, wherein determining the tool alignment based on the second point cloud comprises fitting a line or plane to a set of points in the second point cloud.

8. The method of claim 1, wherein applying the point cloud neural network comprises:
applying an input transform to a first array that comprises the first point cloud to generate a second array, wherein the input transform is implemented using a first T-Net model;
applying a first multi-layer perceptron (MLP) to the second array to generate a third array;
applying a feature transform to the third array to generate a fourth array, wherein the input transform is implemented using a second T-Net model;
applying a second MLP to the fourth array to generate a fifth array;
applying a max pooling layer to the fifth array to generate a global feature vector;
sampling N points in a unit square in 2-dimensions;
concatenating the sampled points with the global feature vector to obtain a combined vector; and
applying one or more third MLPs to generate points in the second point cloud.

9. The method of claim 1, further comprising training the point cloud neural network, wherein training the point cloud neural network comprises:
generating training datasets based on surgical plans of historic patients; and
training the point cloud neural network using the training datasets.

10. A system comprising:
a storage system configured to store a first point cloud representing one or more bones of a patient; and
processing circuitry configured to:
apply a point cloud neural network to generate a second point cloud based on the first point cloud, the second point cloud comprising points indicating a tool alignment; and
determine the tool alignment based on the points indicating the tool alignment.

11. The system of claim 10, wherein the tool alignment is one of: a cutting plane, a drilling axis, or a pin insertion axis.

12. The system of claim 10, further comprising a manufacturing system configured to manufacture a patient-specific tool alignment guide configured to guide a tool along the tool alignment to a target bone of one or more bones of the patient.

13. The system of claim 10, wherein the processing circuitry is further configured to generate, based on the second point cloud, a Mixed Reality visualization indicating the tool alignment.

14. The system of claim 10, wherein the processing circuitry is further configured to control operation of a tool based on alignment of the tool with the tool alignment.

15. The system of claim 10, wherein the second point cloud includes points representing a target bone of the one or more bones of the patient and the points indicating the tool alignment.

16. The system of claim 10, wherein the processing circuitry is configured to, as part of determining the tool alignment based on the second point cloud, fit a line or plane to a set of points in the second point cloud.

17. The system of claim 10, wherein the processing circuitry is configured to, as part of applying the point cloud neural network:
apply an input transform to a first array that comprises the first point cloud to generate a second array, wherein the input transform is implemented using a first T-Net model;
apply a first multi-layer perceptron (MLP) to the second array to generate a third array;
apply a feature transform to the third array to generate a fourth array, wherein the input transform is implemented using a second T-Net model;
apply a second MLP to the fourth array to generate a fifth array;
apply a max pooling layer to the fifth array to generate a global feature vector;
sample N points in a unit square in 2-dimensions;
concatenate the sampled points with the global feature vector to obtain a combined vector; and
apply one or more third MLPs to generate points in the second point cloud.

18. The system of claim 10, wherein the processing circuitry is further configured to train the point cloud neural network, wherein the processing circuitry is configured to, as part of training the point cloud neural network:
generate training datasets based on surgical plans of historic patients; and
train the point cloud neural network using the training datasets.

19-33. (canceled)

34. One or more non-transitory computer-readable storage media having instructions stored thereon that, when executed, cause a computing system to:
obtain a first point cloud representing one or more bones of a patient;
apply a point cloud neural network to generate a second point cloud based on the first point cloud, the second point cloud comprising points indicating a tool alignment; and
determine the tool alignment based on the points indicating the tool alignment.

35. The one or more non-transitory computer-readable storage media of claim 34, wherein the tool alignment is one of: a cutting plane, a drilling axis, or a pin insertion axis.
